(19) United States
(12) Patent Application Publication
(10) Pub. No.: US 2006/0246016 A1
(43) Pub. Date: Nov. 2, 2006

(54) Toothpaste containing anticancer agents

(76) Inventor: Stanislaw R. Burzynski, Houston, TX (US)

Correspondence Address: HOWREY LLP, C/O IP DOCKETING DEPARTMENT, 2941 FAIRVIEW PARK DRIVE, SUITE 200, FALLS CHURCH, VA, 22042-7195, US

(21) Appl. No.: 11/481,542

(22) Filed: Jul. 6, 2006

Related U.S. Application Data
(63) Continuation-in-part of application No. 10/446,536, filed on May 28, 2003, now Pat. No. 7,087,219.

(51) Int. Cl. A61K 8/49

(52) U.S. Cl. 424/49

(57) ABSTRACT

A novel dentifrice composition is provided for prevention or treatment of carcinoma of the oral cavity, caries and periodontal diseases of the oral cavity. The dentifrice composition contains a silica abrasive and medicinal agents useful in the treatment of human neoplastic disease. The medicinal agent is selected from the group consisting of 3-N-phenylacetylamino-2,6-piperidinedione, phenylacetylglutamine, phenylacetylisoglutamine, phenylbutyrate, phenylacetate, combinations thereof and pharmaceutically acceptable salts thereof. The components of the dentifrice composition act advantageously to allow the composition to remove plaque, tartar, and oral disease-causing bacteria.

This application is a continuation-in-part application of U.S. Ser. No. 10/446,536, filed May 28, 2003, the entire contents of which are incorporated by reference herein.

### FIELD OF THE INVENTION

The invention relates to dental and oral prophylactics and, more specifically, to dentifrices such as toothpastes and oral washes which exhibit medicinal, prophylactic, and hygienic effects. In particular, a composition is disclosed for a dentifrice toothpaste containing a silica abrasive including one or more of the following medicinal agents or pharmaceutically acceptable salts thereof: 3-N-phenylacetylamino-2,6-piperidinedione, phenylacetylglutamine, phenylacetylisoglutamine, phenylbutyrate or phenylacetate.

### BACKGROUND OF THE INVENTION

Diseases of the oral cavity are numerous, and include periodontitis, gingivitis, dental caries, halitosis, aphthous ulcers and plaque formation. In many of these diseases, microorganisms have been implicated as causative agents. For example, periodontal diseases, including periodontitis and gingivitis, are caused by bacteria that form plaques on the surfaces of the teeth at the gingival sulcus or pocket. Bacteria known to provoke periodontitis include Actinobacillus actinomycetemcomitans, Porphyromonas gingivalis, Prevotella intermedia, Bacteroides forsytus, and Treponema denticola. Current methods of treatment depend upon the severity of the disease. In almost all cases, regimens of treatment involving a medicated dentifrice and/or an oral rinse are utilized. Mild cases, such as simple calculus (tartar) buildup, are generally treated by the mechanical removal of the irritants. Interproximal cleaning is also important in maintaining gingival health. It is well known that mild cases of periodontal diseases in time progress to become more severe, and even pedantic cleaning with common dentifrices can only prolong the progression of these diseases. The large variety of toothpastes on the market is evidence to this immanent imperfection. More severe cases are generally treated surgically by the removal of gingival tissue, polishing of the tooth roots, or in some rare cases, splinting of the teeth. In the case of bacterial infection from one of the above-cited bacteria, treatment with both antibiotics and surgery becomes necessary. Most of these latter approaches are both painful and costly.

Prophylactic measures can be taken to forestall the occurrence, or recurrence of periodontal diseases. Known prophylactic measures include regular removal of calculus and plaque and the use of dental floss. Such measures are typically time consuming, and involve a strict regimen of care in order to be effective. For this reason, known prophylactic measures are rarely completely effective in preventing diseases such as periodontal disease.

Similarly, oral carcinomas are one of the most prevalent cancers worldwide. According to recent statistics, cancers of the oral cavity and oropharynx account for approximately 3% of all cancers diagnosed in the United States each year. The survival rate of oral carcinomas is about 50%, and deaths due to oral and oropharyngeal cancers make it one of the ten most common causes of death. The majority of oral cancers are squamous cell carcinomas and most involve the tongue, oropharynx, and floor of the mouth, with the lips, gingiva, dorsal tongue, and palate being less common sites.

Typically, surgery and/or radiation therapy are the current treatments of choice for oral cancers. Additionally, chemotherapeutic agents such as cisplatin, bleomycin, 5-FU, docetaxel, interferon alpha, and methotrexate may reduce tumor size and delay metastasis, but to date their use has not had significant positive effects. As a result, most current chemotherapeutic agents are used as adjuncts to standard surgery and/or radiation therapy, and more often in advanced stages of the disease.

Many and varied applications of dentifrice and mouthwash formulations have been described in the patent and medical literature for the use in oral hygiene and for the treatment of periodontal diseases. These formulations generally include a multitude of active ingredients such as fluoride agents, abrasives, and antibacterial agents. Illustrative of these are U.S. Pat. No. 5,374,418, U.S. Pat. No. 5,597,553, U.S. Pat. No. 6,086,372, U.S. Pat. No. 6,123,925, and U.S. Pat. No. 6,331,291.

U.S. Pat. No. 4,915,936 describes a dental hygiene oral rinse composition for the treatment of gingivitis and related periodontal diseases based on a calcium sulfate hemihydrate (beta-form) compound, water as a liquid carrier, alcohol, and other additives. The described formulation further incorporated a humectant to aid in the adherence of the calcium sulfate hemihydrate to teeth and tissue.

Similarly, several patents have suggested formulations for the topical treatment of carcinomas of the oral cavity. For instance, U.S. Pat. No. 5,626,838 describes the use of the NSAID agent ketorolac (5(benzoyl)-2,3-dihydro-1H-pyrrolizine-1-carboxylic acid) for the treatment of primary squamous cell carcinoma, wherein the NSAID (non-steroidal anti-inflammatory drug) agent is in the form of a mouthwash, mouth spray, dental solution/rinse or toothpaste. However, none of these applications have been directed to a composition that would serve as both a prophylactic and a treatment for diseases of the oral cavity and oral carcinomas. Additionally, most of the current formulations described above utilize a variety of compounds that are potentially detrimental to the long-term health of the patients, especially children.

Thus, there exists a need for a dentifrice composition having both oral hygiene and oral carcinoma prophylactic properties, as well as having the ability to be used as a treatment for such maladies. Furthermore, such a dentifrice composition would utilize mostly natural ingredients in order to minimize any long term side effects. The present dentifrice compositions provide these features, and are composed with original, unparalleled components. They are easily prepared from non-toxic, pharmacologically pure materials, and have exhibited effects in the treatment of a variety of oral diseases and hygiene problems. Additionally, it appears that the use of the present dentifrice composition has prophylactic effects with regard to both diseases and carcinomas of the oral cavity.

### SUMMARY OF THE INVENTION

Dentifrice compositions are disclosed containing a silica abrasive including one or more of the medicinal agents 3-N-phenylacetylamino-2,6-piperidinedione, phenylacetylglutamine, phenylacetylisoglutamine, phenylbutyrate or phenylacetate. Pharmaceutically acceptable salts of the aforementioned medical agents can be similarly used. All embodiments of the invention incorporate a silica abrasive, one embodiment of which preferably incorporates silica gel. Invention embodiments may also contain a flavoring oil. The dentifrice compositions have oral hygiene treatment capabilities, as well as prophylactic and therapeutic effects with regard to periodontal disease, caries, and oral carcinomas.

One preferred embodiment of the invention comprises the medicinal agents 3-N-phenylacetylamino-2,6-piperidinedione, phenylacetylglutamine and phenylacetylisoglutamine. Another preferred embodiment comprises 4 parts phenylacetylglutamine to 1 part phenylacetylisoglutamine, and even more preferably, where this particular combination is at a total concentration of 0.1% of the toothpaste composition by weight.

In two other preferred embodiments, the invention incorporates either phenylacetylglutamine, phenylacetylisoglutamine or 3-N-phenylacetylamino-2,6-piperidinedione alone as the medicinal agent, where the concentration of the agent is at about 0.1 wt. % to about 5 wt. % of the total dentifrice composition. The toothpaste composition may preferably contain phenylacetate and/or phenylbutyrate only or in addition to one or more of 3-N-phenylacetylamino-2,6-piperidinedione, phenylacetylglutamine and phenylacetylisoglutamine.

In a further embodiment, the present invention relates to a process of preparing a dentifrice composition comprising a silica abrasive and one or more of the medicinal agents 3-N-phenylacetylamino-2,6-piperidinedione, phenylacetylglutamine, phenylacetylisoglutamine, phenylbutyrate and phenylacetate.. This process provides a dentifrice composition capable of both effectively treating diseases of the oral cavity such as periodontal diseases and caries, and exhibiting a prophylactic effect towards oral carcinomas. The compositions are typically applied manually to the desired areas of the oral cavity, preferably in the morning and in the evening. Preliminary studies with the dentifrice compositions of the present invention demonstrate a significant reduction in diseases of the oral cavity such as calculus buildup, caries, gingivitis, and other periodontal disorders. An unexpected result from these preliminary studies was the removal of tobacco- and/or tea and coffee-related teeth stains using the dentifrice compositions described herein, without the need for incorporating harsh abrasives or peroxide stain removers. Further, the present invention has been shown to exhibit anti-cancer activity in the oral cavity and renewal of damaged epithelium gum tissue in the oral cavity.

### DETAILED DESCRIPTION OF THE PREFERRED EMBODIMENTS

The present invention provides dentifrice compositions for alleviating or preventing diseases and/or carcinomas of the oral cavity. The invention is described hereafter in terms of preferred embodiments known at the time of the application. These embodiments represent the best mode contemplated for formulating and preparing the dentifrice compositions of the present invention.

As used herein, term “dentifrice” or “dentifrices” is intended to refer to products which remain in the mouth for a relatively short period of time, in which they are intimately contacted with substantially all surfaces of the teeth, and are then removed. Non-limiting examples of such products include toothpastes, prophylactic pastes, tooth polishes, gels, professional gels and other related products applied by dentists, as well as mouth washes, mouth rinses, dental flosses, chewing gums, lozenges, tablets, edible food products, and the like.

In one preferred embodiment, the compositions of the dentifrice of the present invention are toothpastes, mouth rinses, mouth sprays, and dental solutions. Preferably, the composition is toothpaste or a mouth rinse, and more preferably the dentifrice composition is toothpaste.

Hereafter, whenever the terms 3-N-phenylacetylamino-2,6-piperidinedione, phenylacetylglutamine, phenylacetylisoglutamine, phenylacetate and phenylbutyrate are used, they should be viewed as encompassing both the acidic and pharmaceutically acceptable salt forms of the molecules.

3-N-phenylacetylamino-2,6-piperidinedione has the structural formula provided below as Formula I.

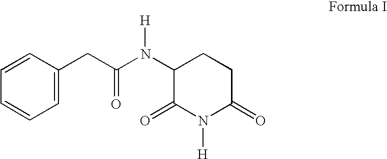

Phenylacetylglutamine has a molecular weight of 264.28, its empirical formula is C₁₆H₁₆N₂O₄, and the structural formula provided below as Formula II.

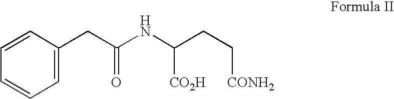

Phenylacetylisoglutamine has a molecular weight of 264.28; its empirical formula is C₁₆H₁₆N₂O₄, and the structural formula provided below as Formula III.

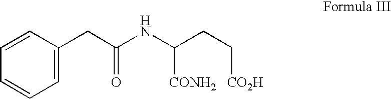

Phenylacetate has a molecular weight of 136.14, its empirical formula is C₈H₈O₂, and the structural formula is provided below as Formula IV.

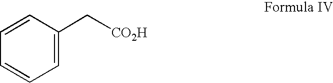

Phenylbutyrate has a molecular weight of 164.2, its empirical formula is C₁₀H₁₂O₂, and the structural formula is provided below as Formula V (sodium salt as shown).

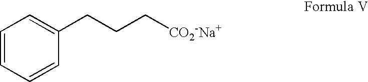

The preferred medicinal agent for use with the dentifrice of the present invention for the prevention and treatment of periodontitis, caries, oral disease, and carcinomas of the oral cavity or oropharynx is the amino acid analogue 3-N-phenylacetylamino-2,6-piperidinedione (3-[N-phenylacetylaminopiperidine]-2,6-dion]), also known as antineoplaston A10, and its pharmaceutically acceptable salts. However, other related piperidine dione derivatives which are related to 3-N-phenylacetylamino-2,6-piperidinedione are also envisioned to be useful, such as those described in U.S. Pat. No. 5,238,947, which is incorporated herein in its entirety, as well as the pharmaceutically acceptable salts thereof.

While 3-N-phenylacetylamino-2,6-piperidinedione is a useful drug in the treatment of neoplastic diseases, its incorporation into a dentifrice composition is unknown prior to the present invention being made. According to numerous publications and patents related to this family of compounds (See, for example, Burzynski, S. R., et al., Drugs Exptl. Clin. Res. 12 Suppl., 1, 25-35 (1986);), 3-N-phenylacetylamino-2,6-piperidinedione has been shown to be effective in treating a variety of cancers in clinical trials. It beneficially is a completely non-toxic compound, and has even appeared on the market as a food supplement. The addition of 3-N-phenylacetylamino-2,6-piperidinedione to the dentifrice composition of the present invention may provide the composition with prophylactic characteristics with regard to oral carcinomas. Nonetheless, the incorporation of 3-N-phenylacetylamino-2,6-piperidinedione to a dentifrice composition gave the compositions the needed physical properties, e.g. smoothness, needed.

3-N-Phenylacetylamino-2,6-piperidinedione can be isolated from human body fluids, such as urine, as described in U.S. Pat. No. 4,558,057. Alternatively, it can be synthesized by reacting the amino acid L-glutamine with a phenylacetyl halide in a weakly alkaline aqueous solution, lowering the pH of the solution, and subsequently heating the mixture under vacuum at 160° C. to yield 3-N-phenylacetylamino-2,6-piperidinedione, as described in U.S. Pat. No. 4,918,193 and U.S. Pat. No. 4,470,970, both of which are herein incorporated by reference.

As described herein, “pharmaceutically acceptable salts” means salts having the biological activity of the parent compound and lacking toxic activity at the selected administration level. Again, determination of whether a salt is pharmaceutically acceptable can be accomplished by methods known to those of skill in the art. Pharmaceutically acceptable salts of 3-N-phenylacetylamino-2,6-piperidinedione include, but are not limited to, inorganic sodium, potassium and ammonium salts, and organic diethanolamine, cyclohexylamine, and amino acid salts. Preferably, the salt is a sodium salt.

Suitable acids for forming acid addition salts of the compounds of the present invention include, but are not limited to, acetic, hydrochloric, citric, fumaric, gluconic, glucuronic, glutamic, lactic, malic, maleic, pamoic, salicylic, stearic, succinic, and tartaric acids. The class of acids suitable for the formation of nontoxic, pharmaceutically-acceptable salts is well known to practitioners of the pharmaceutical formulation arts. (See, e.g., Stephen N. Berge, et al., Journal of Pharmaceutical Sciences, Vol. 66; pp. 1-19 (1977); and, P. Heinrich Stahl, et al., Handbook of Pharmaceutical Salts: Properties, Selection, and Use, Wiley-VCH, 2002).

Further, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

In the present invention, the amount of antineoplaston A10 (3-N-phenylacetylamino-2,6-piperidinedione) present in the dentifrice composition can be in the range of about 0.1% by weight to about 5.0% by weight. Preferably, the 3-N-phenylacetylamino-2,6-piperidinedione is present in the range of about 0.4% by weight to about 4.5% by weight.

In addition to the abovementioned use of 3-N-phenylacetylamino-2,6-piperidinedione as a medicinal agent in the present invention, its downstream metabolites, phenylacetylglutamine and phenylacetylisoglutamine, can similarly be incorporated in the dentifrice composition. One preferred embodiment thereof incorporates phenylacetylglutamine alone as the medicinal agent, whereas another preferably incorporates both phenylacetylglutamine and phenylacetylisoglutamine in a ratio of 4:1. Phenylacetylglutamine and phenylacetylisoglutamine are the hydrolysis products, formed in the ratio of 4:1, resulting from salt-neutralization of 3-N-phenylacetylamino-2,6-piperidinedione. They are also the downstream products of 3-N-phenylacetylamino-2,6-piperidinedione metabolism in vivo. Both these metabolites are postulated to play a role in providing the cancer-suppressing activity of 3-N-phenylacetylamino-2,6-piperidinedione and may provide such an effect in the oral cavity.

The present invention may also incorporate the medicinal agents phenylacetate and phenylbutyrate, which are upstream metabolites of phenylacetylglutamine. Both these components may provide anti-cancer activity to the dentifrice composition through their direct inhibitory action on cancer cells (e.g. stimulation of cellular differentiation) as well as their role in phenylacetylglutamine synthesis.

Silica abrasives are also incorporated into the dentifrice composition of the present invention. Specific silica abrasives suitable for use with the present invention include but are not limited to silica gels, precipitated silicas, silicates, and hydrated silica. Silica gels suitable for use with the present invention are hydrogels, hydrous gels, xerogels, and aerogels, such as those known in the art and described in U.S. Pat. No. 6,440,397, which is incorporated herein by reference. Precipitated silicas are those known in the art, such as the suitable dentifrice-type precipitated silicas described in U.S. Pat. No. 5,589,160, the contents of which are incorporated by reference. Suitable silicates are any of those naturally occurring or synthetic silicates suitable for use with dentifrice compositions. These silica abrasives can be used singly or in combination. The preferred silica abrasive for use with the present invention includes silica gels. The silica abrasives can be used together with the calcium salt or in lieu of the calcium salt component.

The silica abrasive is desirably incorporated into the dentifrice composition of the present invention, such as toothpastes, tooth gels, mouth rinses, and the like in an amount ranging from about 10% to about 80% by weight of the dentifrice composition. Preferably, about 35% to about 70% by weight of the dentifrice toothpaste composition is a silica abrasive paste or gel.

An optional ingredient in dentifrice formulations of the present invention is a calcium salt having at least partial water solubility, such as calcium sulfate dihydrate (gypsum, CaSO₄.2H₂O), pharmaceutical grade. By “at least partial water solubility”, it is meant that the calcium salt should have a solubility of at least 0.10 g/100 mL water at 25° C., or a solubility product constant (ksp) of at least about 3.1×10⁻⁷ to about 5.0×10⁻⁵ at 25° C. In the event that calcium sulfate is used, the calcium sulfate may be from any natural source, such as gypsum rock, preferably calcined and meeting both FDA and Codex requirements as to purity. Calcium sulfate synthesized from various chemical processes known to those of skill in the art may also be used, such as the by-product chemical gypsum from citric acid manufacture. Additional, non-limiting examples of calcium salts of at least partial water solubility suitable for use in this invention include calcium sulfate, anhydrous calcium sulfate, calcium sulfate hemihydrate (CaSO₄.½H₂O), calcium malate, calcium tartrate, calcium lactate, calcium mesoxalate, calcium malonate, calcium succinate, calcium glycerophosphate, and mixtures of the foregoing. Preferably, calcium sulfate dihydrate is used as the calcium salt component.

A further component of the dentifrice composition of this invention can be hydrophilic liquid vehicles, including but not limited to glycerin, propylene glycol, polyethylene glycol, and hydrophobic liquid vehicles such as triglyceride, diglyceride, and organic oils including mineral oil, essential oils, and fatty vegetable oils. Essential oils, as used herein, are natural substances which are extracted via distillation from tiny molecular sacs of each botanical, and are part of the plant's immune system and yet a separate substance created from the plant. The distillation process extracts the volatile oil from the plant parts. Such essential oils are highly concentrated extracts that contain hormones, vitamins, and antiseptics that work on many levels. Preferably, the liquid vehicle is mineral oil. These hydrophilic and hydrophobic liquid vehicles can be used either singly or in combination and preferably, can be added in a proportion of from about 2 to about 50 wt. % (in the case of compositions comprising liquid vehicles), especially from about 10 to about 35 wt. % based on the whole composition.

Using one or more of these liquid vehicles, the composition of the present invention for the oral cavity may preferably be formulated into a use form such as gel, liquid, or paste.

The dentifrice composition of the present invention also contains flavor components, typically in the form of natural flavors or aroma oils and/or herbal extracts and oils. These flavor components can serve not only to give a palatable flavor to the dentifrice composition, but can act as natural antibacterial agents and preservatives at the same time. The oils suitable for use in the present invention include but are not limited to citric oil, lemon oil, lime oil, lemongrass oil, orange oil, sweet orange oil, grapefruit oil, pomegranate oil, apricot oil extract, tangerine extract, tangelo oil, peppermint oil, spearmint oil, sage oil, rosemary oil, cinnamon oil, winter green oil, clove oil, eucalyptus oil, ginger oil, sassafras oil, menthol, arvensis mint oil, synthetic mint flavors and oils, carvone, eugenol, methyleugenol, methyl salicylate, methyl eugenol, thymol, anethole, millefolium extract, chamomile, lavender oil, myrrh, eugenol, tea tree oil, sage oil, mallow, limonene, ocimene, n-decyl alcohol, citronellol, α-terpineol, linalol, ethyllinalol, thyme, almond oil, nutmeg, and vanillin. Either one of these flavors or a mixture of two or more of these flavors can be used in the dentifrice composition. The content thereof ranges from about 3% to about 20% by weight, preferably from about 4% to about 15% by weight, based on the whole composition.

Water can optionally be incorporated into the present toothpaste compositions of the present invention. Water used in the preparation of commercially suitable toothpastes should preferably be deionized and free of organic impurities. Water can generally comprise about 0% to about 40% by weight of the toothpaste compositions herein.

In addition to the above-described essential components, the dentifrice composition of the present invention can further contain a variety of optional ingredients and vehicles generally used for preparations for use in the oral cavity, such as dentifrices. These optional components include, but are not limited to, such components as abrasives, surfactants, thickening agents, buffers, humectants, preservatives, and antibiotic and anti-caries agents. All of these additives, described in further detail below, are generally usual and would be known to one of skill in the art.

Dental abrasives useful in the dentifrice compositions of the present invention include a variety of different materials known in the art. Preferably, the abrasive material should be one which is compatible with the composition of interest and does not excessively abrade dentin. Suitable abrasives include for example, silicas including gels and precipitates; insoluble polymetaphosphate, hydrated alumina, resinous abrasives such as polymerized resins (e.g. ureas, melamines, cross-linked epoxides, phenolics, and the like), and mixtures thereof.

Another optional component of the dentifrice compositions of the present invention is a humectant. The humectant serves to keep compositions such as toothpaste compositions from hardening upon exposure to air, and to give mouthwash and toothpaste compositions a moist feel to the mouth. Certain humectants can also impart desirable sweetness of flavor to toothpaste and mouthwash compositions. Suitable humectants for use in compositions of the present invention include edible polyhydric alcohols such as glycerin, sorbitol, xylitol, polyethylene glycol, and propylene glycol.

The dentifrice compositions of the present invention can also optionally contain sweeteners such as saccharin sodium, acesulfame potassium, glycyrrhizin, perillartine, thaumatin, aspartylphenylalanyl methyl ester and xylitol.

Buffering agents are another optional component of the dentifrice compositions of the present invention. The buffering agents serve to retain the pH of the compositions within the preferred range. Suitable buffering agents for use in dentifrice compositions of the present invention include soluble phosphate salts.

Other optional components of the dentifrice compositions of the present invention are preservatives, such as those that prevent microbial growth in the dentifrice compositions. Suitable preservatives include but are not limited to methylparaben, propylparaben, bezoates, and alcohols such as ethanol.

Binders and thickeners can also optionally be used in the dentifrice compositions of the present invention, particularly in toothpaste compositions. Preferred binders and thickening agents include, but are not limited to, carrageenan (e.g. Viscarin, Irish moss, and the like); cellulose derivatives such as hydroxyethyl cellulose, sodium carboxymethyl cellulose, and sodium carboxymethyl hydroxypropyl cellulose, carboxyvinyl polymers; natural gums such as karaya gum, gum Arabic, and tragacanth; polysaccharide gums such as xanthan gum; fumed silica; and colloidal magnesium aluminum silicate.

Compositions of the present dentifrice compositions can also optionally contain a surfactant. Suitable surfactants are those which are reasonably stable and preferably form suds (bubbles) throughout the pH range of the dentifrice compositions. Surfactants can also be added to act as solubilizing agents to help retain sparingly soluble components in solutions or mixtures. Surfactants useful in the dentifrice compositions as sudsing agents can be soaps, polysorbates, poloxamers, and synthetic detergents that are anionic, nonionic, cationic, zwitterionic, or amphoteric, and mixtures thereof.

The dentifrice compositions of the present invention can also optionally comprise anti-caries agents. Preferred anti-caries agents are water-soluble fluoride ion sources. The number of such fluoride ions sources is great and well known to those of skill in the art, and includes those disclosed in U.S. Pat. No. 3,535,421, which is incorporated herein by reference. Preferred fluoride ion source materials include sodium fluoride, potassium fluoride, sodium monofluorophosphate and mixtures thereof.

Antimicrobial and anti-plaque agents can also optionally be present in the dentifrice compositions of the present condition. Such agents may include: triclosan (5-chloro-2-(2,4-dichlorophenoxy)-phenol); chlorhexidine; chlorhexidine digluconate (CHX); alexidine, hexetidine (HEX); sanguinarine (SNG); benzalkonium chloride; salicylanilide; domiphen bromide; cetylpyridiniumchloride (CPC); tetradecylpyridinium chloride (TPC); N-tetra-decyl-4-ethylpyridinium chloride (TDEPC); octenidine; delmopinol; octapinol, and other piperidino derivatives; nicin preparations; zinc/stannous ion agents; antibiotics such as augmentin, amoxicillin, tetracycline, deoxycycline, minocycline, and metronidazole; peroxide, such as cylium peroxide, hydrogen peroxide, and magnesium monoperthalate an its analogs; and analogs and salts of the above listed antimicrobial and antiplaque agents.

Dentifrice compositions of the present invention can also optionally include one or more anticalculus (anti-tartar) agents. Anticalculus agents which may be useful in the dentifrice compositions of the present invention include antimicrobials such as chlorhexidine, niddamycin, and triclosan, metals and metal salts such as zinc citrate, Vitamin C, bisphosphonates, triclosanpyrophosphates, pyrophosphates, polyphosphates, polyacrylates and other polycarboxylates, polyepoxysuccinates, ethylenediaminetetraacetic acid (EDTA), nitrilotriacetic acid and related compounds, polyphosphonates, and polypyrophosphates such as sodium hexametaphosphate, as well as other anticalculus agents known to those of skill in the art, such as those described in K. J. Fairbrother et al., “Anticalculus agents,” Journal of Clinical Periodontology Vol. 27, pp. 285-301 (2000).

Nutrients and vitamins can also optionally be added to the dentifrice compositions of the present invention. Such agents can include folates, retinoids (Vitamin A), Vitamin B (B₁-thyamin, B₂-riboflavin, B₃-niacine, B₅-pantothenic acid, B₆-pyridoxine, B₇-biotin, B₈/B₉/Bc-folic acid, B₁₂-cyanocobalamin), Vitamin C (ascorbic acid, sodium ascorbate), Vitamin E, Vitamin E analogs (d1-α-tocopherol acetate, tocopherol succinate, tocopherol nicotinate)and zinc.

A variety of miscellaneous additives can also be optionally formulated into the dentifrices of the present invention, such as tooth desensitizing agents (e.g. potassium and strontium salts), condensed anti-tartar agents such as sodium and potassium tetrapyrophosphate, whitening agents such as aluminum oxide and calcium peroxide, debriding agents such as sodium bicarbonate, pigments and dyes, such as Blue 15-C174160, Green 7-C174260, Reds 4-CI12085 and 40 CI16035, Yellows 115 CI47005:1 and 5 CI19140, and Carnine 5 CI16035), as well as additives such as mica and sparkles. As with the other optional dentifrice additives, use can be made of either one of these ingredients or a mixture of two or more of these ingredients in amounts appropriate for the dentifrice composition.

An unexpected cleaning performance of this invention has also been demonstrated. The dentifrice of the present invention has surprisingly exhibited the ability to remove tar deposits related to tobacco use from the teeth and other surfaces of the oral cavity, as well as stains on the teeth due to coffee and/or tea consumption. It is well known that the use of tobacco products deposits a constituent commonly referred to as “tar” within the oral cavity. Tobacco tar, as used herein, is loosely defined as a dark, oily, viscous blend of polycyclic aromatic and aliphatic hydrocarbons, as well as other compounds. Tar is produced as tobacco in a cigarette, cigar, or pipe is burned, and as tobacco is chewed. This tar readily forms a solution with saliva, and enzymes in the saliva assist in the degradation of tobacco itself. The tobacco and saliva-tar solution, as well as materials contained therein, can be deposited on the surfaces within the oral cavity of the tobacco users, leaving resinous stains.

Due to the hydrophobic nature of compounds contained within the tar, the tar is not easily dissolved, solubilized, detached, and/or dispersed by commonly available, over the counter dentifrices such as toothpastes, gels, and oral rinses. As a result, tobacco tar can buildup on the teeth and other surfaces of tobacco users' oral cavities, causing chronic halitosis, increased calculus (tartar) buildup, and an increased incidence of periodontal diseases, as wall as unsightly staining.

Prior to the present invention, the known anti-tobacco/tar stain products on the market were dentifrices such as toothpastes which did not contain ingredients with the ability to dissolve, solubilize, or otherwise remove tobacco stains or tar, but rather, removed tobacco stains and tar deposits through the presence of physical abrading agents. Unfortunately, such abrading materials, which are strong enough to remove tobacco stains and tar buildup, also tend to abrade tooth enamel and can promote tooth decay and farther oral diseases.

It has been unexpectedly found that the dentifrice composition of the present invention has the ability to reduce tar buildup and stains due to tobacco use, as well as stains due to coffee and/or tea consumption, without the use of a strong abrasive component. As indicated above, the combination of a slightly water soluble calcium compound with a liquid vehicle and the antineoplaston A10 (3-N-phenylacetylamino-2,6-piperidinedione) provides a composition that easily emulsifies with saliva in the mouth and readily cleans the surfaces of the oral cavity without the use of harsh abrasives. Patients who were tobacco users, coffee and/or tea consumers, or both, and who used a toothpaste having the composition of the present invention exhibited noticeable stain removal.

The composition according to the present invention may preferably be used by applying it into the oral cavity and brushing the teeth and/or gingivae. For such brushing, use of a toothbrush provided with plaque removing bristles and gingiva-massaging portions in combination is especially preferred, although a conventional toothbrush, namely, a toothbrush having plaque removing bristles or a toothbrush having massaging portions may also be used.

In the process for preparing the dentifrice toothpaste of the present invention, the first step is the preparation of the oil component. In the preparation of the oil component, one or more natural flavor or aroma oils are added to a volume of a liquid vehicle, such as paraffin oil. The mixture is stirred, either mechanically or by hand, for a time sufficient to form a homogenous mixture. It is generally preferred that the temperature of the mixture be maintained at or near ambient temperature during the preparation.

For the preparation of the toothpaste, the homogenous oil mixture is stirred with a) an appropriate slightly water-soluble calcium salt or a mixture of calcium salts; b) 3-N-phenylacetylamino-2,6-piperidinedione; and c) optionally, water or other vehicle, (e.g. glycerin), in order to obtain a complete toothpaste formulation. In a preferred method of preparation, the flavor and aroma oils are added together and then brought up to the desired volume with the liquid vehicle. This flavor oil/vehicle solution is then stirred together until the mixture is homogenous. Thereafter, the calcium salt and 3-N-phenylacetylamino-2,6-piperidinedione are ground together to form a homogenous powder. This homogenous powder is then stirred together, mechanically or by hand, with the oil mixture until a smooth paste forms. The paste can then be packed into tubes in a manner known to those of skill in the art.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

Toothpaste Formulation Containing Calcium Sulfate Dihydrate and about 0.5 wt. % of 3-N-phenylacetylamino-2,6-piperidinedione.

A toothpaste formulation according to the present invention was prepared as follows:

To an appropriate measuring container was added 6 mL of grapefruit oil, 2 mL of citric oil, 2 mL of sweet orange oil, 2 mL of peppermint oil, and 2 mL of eucalyptus oil, and the flavor oils mixed at ambient temperature. Thick liquid paraffin [Food Grade] (also known as mineral oil) was then added in an amount sufficient to bring the total volume of the mixture up to 100 mL. The oil component was mixed so as to form a homogenous solution. This is the base flavoring oil component for use in the toothpaste examples described in Examples 2-5.

To 30 g of finely powdered gypsum (calcium sulfate dihydrate; pure for the food production industry) was added 0.2 g 3-N-phenylacetylamino-2,6-piperidinedione as a dry, solid powder. The two solids mixed to form a homogenous solid, and then 12 g of the flavoring oil component from above was added. The composition of the powders and oils was mixed together to form a smooth paste. The paste was then packed into a tube.

### Example 2

Toothpaste Formulation Containing Calcium Sulfate Dihydrate and about 5.0 wt. % of 3-N-phenylacetylamino-2,6-piperidinedione.

The paste composition in Example 1 was reformed, this time using 2.2 g of 3-N-phenylacetylamino-2,6-piperidinedione, in the same manner as described. The paste was formed into a smooth paste, as before, and packed into a tube. The toothpaste of this formulation had a slightly bitter taste.

### Example 3

Toothpaste Formulation Containing Calcium Sulfate Dihydrate about 5.0 wt. % of 3-N-phenylacetylamino-2,6-piperidinedione and glycerin.

To 60 g of finely powdered gypsum (as in Example 1) was added 1.2 g of 3-N-phenylacetylamino-2,6-piperidinedione as a dry powder, and the two solids were mixed together to form a homogenous powder. This solid powder was then mixed with 32 g of the oil component (as prepared in Example 1) for 30 minutes. A smooth, half-liquid paste results. To this paste was added 4 g of glycerin (available from numerous commercial sources), and the mixing continued for a further 30 minutes. The product paste was packed into small (20 g) aluminum tubes, and was ready for use. The toothpaste of this formulation had a pleasant, sweet taste.

### Example 4

Clinical Study of the Calcium Sulfate Dentifrice Composition.

In a test of examination of the stability of the new paste, 30 test subjects varying in age from 15 to 65, both genders, used the toothpaste prepared according to either Example 1 or Example 2 over a one month period as their sole toothpaste. The individuals cleaned their teeth two times daily, once in the morning and again in the evening following their last meal. The individuals were under dentist supervision throughout, as well as during the initial and final examinations. None of the individuals reported intolerance or hypersensitivity to the new toothpastes. In all of the test subjects, the dental examiner found significant reduction of plaque. All subjects reported clean teeth and improved mouth hygiene all day. Those test subjects who were tobacco users (smokers, chewing tobacco) or heavy tea/coffee drinkers exhibited none of the stains typically found on the teeth that are consistent with tobacco or coffee/tea use.

### Example 5

Clinical Treatment of Oral Disease with the Calcium Sulfate-Based Dentifrice Composition.

Four adult individuals with advanced periodontitis, gum bleeding, and plaque buildup were given toothpaste formulated according to Example 1 for use two times daily, after meals. After several days, the gum bleeding had stopped. After two weeks of use, the stony plaque buildup on the tooth surface had diminished. Dental examination confirmed the arrest of the periodontitis progress in all four adults.

### Example 6

Toothpaste Formulation Containing Silica Abrasive and about 0.5 wt. % of 3-N-phenylacetylamino-2,6-piperidinedione.

A toothpaste formulation according to the present invention could be prepared as follows:

To 30 g of food-grade silica gel is added 0.2 g 3-N-phenylacetylamino-2,6-piperidinedione as a dry, solid powder. The two solids are mixed to form a homogenous solid, and then 12 g of the flavoring oil component from Example 1 is added. The composition of the powders and oils are mixed together to form a smooth paste, afterwhich the paste is packed into a tube.

### Example 7

Toothpaste Formulation Containing Silica Abrasive and about 1 wt. % of 3-N-phenylacetylamino-2,6-piperidinedione, about 1 wt. % of phenylacetylglutamine, and about 1 wt. % phenylacetylisoglutamine.

The paste composition in Example 6 is reformed in the same manner as described, this time using 0.43 g of 3-N-phenylacetylamino-2,6-piperidinedione, as well as 0.43 g of phenylacetylglutamine and 0.43 g of phenylacetylisoglutamine. The paste is formed into a smooth paste, as above, and packed into a tube. Optionally, this toothpaste formulation may only include phenylacetylglutamine and phenylacetylisoglutamine as the added medicinal agents.

### Example 8

Toothpaste Formulation Containing Silica Abrasive and phenylacetylglutamine and phenylacetylisoglutamine at a Ratio of about 4:1. Respectively.

The paste composition in Example 6 is reformed in the same manner as described, this time using 0.8 g of phenylacetylglutamine and 0.2 g of phenylacetylisoglutamine. The paste is formed into a smooth paste, as above, and packed into a tube.

### Example 9

Toothpaste Formulation Containing Silica Abrasive and about 1 wt. % of phenylbutyrate or phenylacetate.

The paste composition in Example 6 is reformed in the same manner as described, this time using 0.43 g of sodium phenylbutyrate or sodium phenylacetate. The paste is formed into a smooth paste, as above, and packed into a tube. Optionally, this toothpaste formulation may include both phenylbutyrate and phenylacetate at about 1 wt. % each.

### Example 10

Clinical Study of the Silica Gel Dentifrice Composition.

In a test of the new toothpaste, 30-50 test subjects varying in age from 15 to 65, both genders, would use the toothpaste prepared according to one of Examples 6-9 over a one month period as their sole toothpaste. The individuals would clean their teeth two times daily, once in the morning and again in the evening following their last meal. The individuals would be under dentist supervision throughout the study, as well as during the initial and final examinations. It is expected that none of the individuals would report intolerance or hypersensitivity to the silica gel-based toothpaste. It is further expected that the dental examiner would find significant reduction of plaque, in addition to a reduction in stains caused by smoking, chewing tobacco and/or excessive tea/coffee drinking.

### Example 11

Clinical Treatment of Oral Disease with the Silica-Based Dentifrice Composition.

A number of adult individuals with advanced periodontitis, gum bleeding, and plaque buildup would be provided toothpaste formulated according to one of Examples 6-9 for use two times daily, after meals. It is expected that gum bleeding would stop after several days of using the toothpaste. It is further expected that, after two weeks of toothpaste use, stony plaque buildup would have diminished and that the progression of periodontitis would have arrested.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention.

What is claimed is:

1. A toothpaste composition comprising:
a silica abrasive, and
a medicinal agent selected from the group consisting of 3-N-phenylacetylamino-2,6-piperidinedione, phenylacetylglutamine, phenylacetylisoglutamine, phenylbutyrate, phenylacetate, combinations thereof and pharmaceutically acceptable salts thereof.

2. The toothpaste composition of claim 1, wherein the silica abrasive is selected from the group consisting of silica gels, precipitated silicas, silicates, and hydrated silica.

3. The toothpaste composition of claim 2, wherein the silica gel is selected from the group consisting of hydrogels, xerogels, and aerogels.

4. The toothpaste composition of claim 1, wherein the medicinal agent comprises 3-N-phenylacetylamino-2,6-piperidinedione, phenylacetylglutamine and phenylacetylisoglutamine, or pharmaceutically acceptable salts thereof.

5. The toothpaste composition of claim 1, wherein the medicinal agent comprises phenylacetylglutamine and phenylacetylisoglutamine, or pharmaceutically acceptable salts thereof.

6. The toothpaste composition of claim 5, wherein the medicinal agent comprises phenylacetylglutamine and phenylacetylisoglutamine, or pharmaceutically acceptable salts thereof, at a ratio of 4 to 1, respectively.

7. The toothpaste composition of claim 1, wherein the medicinal agent comprises phenylbutyrate or phenylacetate, or a combination thereof or pharmaceutically acceptable salts thereof.

8. The toothpaste composition of claim 1, wherein the medicinal agent comprises phenylacetylglutamine or phenylacetylisoglutamine or pharmaceutically acceptable salts thereof.

9. The toothpaste composition of claim 1, wherein the medicinal agent is at a concentration of about 0.1 wt. % to about 5 wt. % of the total composition.

10. The toothpaste composition of claim 1, wherein the composition contains at least one flavoring oil.

11. A method for providing prophylactic, therapeutic and cosmetic oral care comprising the steps of:
introducing into the oral cavity the toothpaste composition according to claim 1, and
brushing surfaces in the oral cavity with a toothbrush.
